SEQUENCE LISTING (3 sequences)

SEQ ID NO: 1 - protein, 30 aa, Artificial Sequence
  Chemically synthesized peptide
HXEGTFTSDVSSYLEGQAAKEFIAWLVKXR

SEQ ID NO: 2 - protein, 39 aa, Artificial Sequence
  Chemically synthesized Peptide
HGEGTFTSDLSKQMEEEAVRLFIEWLKNGGPSSGAPPPS

SEQ ID NO: 3 - protein, 44 aa, Artificial Sequence
  Chemically synthesized peptide
HGEGTFTSDLSKQMEEEAVRLFIEWLKNGGPSSGAPPSKKKKKK

(19) United States
(12) Patent Application Publication
(10) Pub. No.: US 2011/0313131 A1
(43) Pub. Date: Dec. 22, 2011

(54) REVERSED PHASE HPLC PURIFICATION OF A GLP-1 ANALOGUE

(76) Inventors: Christelle Carl, Blotzheim (FR); Michael Rothe, Loerrach (DE); Christian Saladin, Thuernen (CH); Daniel Strub, Breitenbach (CH); Francis Vix, Riedisheim (FR)

(21) Appl. No.: 13/158,500

(22) Filed: Jun. 13, 2011

(30) Foreign Application Priority Data
Jun. 21, 2010 (EP) 10166602.2

(51) Int. Cl. C07K 14/00; C07K 1/20

(52) U.S. Cl. 530/324; 530/344

(57) ABSTRACT

The invention comprises a process for the purification of a GLP-1 peptide analogue applying reversed phase high performance liquid chromatography (RP-HPLC).

### PRIORITY TO RELATED APPLICATION(S)

This application claims the benefit of European Patent Application No. 10166602.2, filed Jun. 21, 2010, which is hereby incorporated by reference in its entirety.

### FIELD OF THE INVENTION

The invention refers to the purification of analogues of human glucagon-like peptide-1 (GLP-1), particularly to a process for the purification of the GLP-1 analogue with the amino acid sequence according to SEQ ID No. 1:

His-Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Aib-Arg-NH₂, wherein 26 of these amino acids are in the natural L configuration while four are not chiral. Aib means α-aminoisobutyric acid analogues of human glucagon-like peptide-1 (GLP-1) by reversed phase high performance liquid chromatography (RP-HPLC).

This peptide is also named (Aib^{8,35})GLP-1(7-36)NH₂ and its pharmaceutical use and preparation by solid phase peptide synthesis (SPPS) is described in the PCT Publication WO 2000/34331.

### BACKGROUND OF THE INVENTION

The synthesis of GLP-1 analogues can follow a hybrid approach encompassing both solid phase peptide synthesis (SPPS) and fragment couplings in solution. For example the PCT Publication WO 2007/147816 describes the preparation of (Aib^{8,35}) GLP-1(7-36)NH₂ by preparing three fragments and coupling these fragments in solution.

The individual synthetic steps usually are highly selective, however, at the end of a multi-step chemical synthesis the product is typically not pure enough to be used as a drug. The crude product can therefore be subjected to reversed phase high performance liquid chromatography (RP-HPLC), to further purify the peptide and to achieve purity in the range of 96 to 99% (area). After the RP-HPLC stage the product is normally obtained in the form of a solution with a concentration of typically 1 to 15% (w/w) of the peptide.

In order to obtain a dry final product which is suitable for the drug formulation the solution can either be subjected to precipitation, lyophilization or spray-drying techniques.

RP-HPLC purification for human glucagon-like peptide-1 (GLP-1) has been widely described in the art.

For instance according to the PCT Publication WO 2007/147816 the GLP-1 analogue is subjected to a two step RP-HPLC process;

a first chromatography at a pH 2 applying as mobile phases a mixture A consisting of acetonitrile (15%), water (85%) and small amounts of TFA, and a mixture B composed of tetrahydrofuran (15%), acetonitrile (70%), water (15%) and small amounts of TFA and a second chromatography at pH 8.8 applying as mobile phases a mixture A consisting of acetonitrile (15%), water (85%) and ammonium acetate buffer, and a mixture B composed of tetrahydrofuran (15%), acetonitrile (60%), water (25% and ammonium acetate buffer. Since tetrahydrofuran tends to form peroxides the eluent is critical for a RP-HPLC on a large scale.

EP-B1 1664 109 discloses a RP-HPLC method for purifying glucagon like peptides with a pH-buffered alcohol, particularly with ethanol as eluent, whereby the pH range may be set between pH 4 and pH 10, but may not vary from the pH setpoint by more than +/−1.0 pH units. In order to achieve the desired purity the method thus requires strict pH control.

However, it was found that with ethanol as eluent the desired purity could not be achieved, particularly the impurity des-Ser¹⁷, Ser¹⁸-[Aib^{8,35}]hGLP-1(7-36)NH₂ could not be removed efficiently.

The object of the present invention therefore is to develop a RP-HPLC process which is easily applicable on a technical scale, which is safe regarding the solvents and which is able to provide a GLP-1 solution with excellent purity.

It was found that this object could be reached with the process of the present invention as outlined below.

### BRIEF DESCRIPTION OF THE FIGURES

FIG. 1a: RP-HPLC chromatogram of 2^{nd} chromatography of (Aib^{8,35})GLP-1(7-36)NH₂; 20 mM Ammonium acetate, pH=9.2; Kromasil C18 100-16; Ethanol (100%).

FIG. 1b: RP-HPLC chromatogram of 2^{nd} chromatography of (Aib^{8,35})GLP-1(7-36)NH₂; 20 mM Ammonium acetate, pH=9.5; Kromasil C18 100-16; Acetonitril (100%).

Compared to FIG. 1a) the impurity des-Ser¹⁷,Ser¹⁸-[Aib^{8,35}]hGLP-1(7-36)NH₂ was efficiently removed with Acetonitrile as eluent.

FIG. 2a: RP-HPLC chromatogram of 2^{nd} chromatography of (Aib^{8,35})GLP-1(7-36)NH₂; 20 mM Ammonium acetate, pH=9.5; Kromasil C18 100-16; Acetonitrile (100%).

FIG. 2b: RP-HPLC chromatogram of 2^{nd} chromatography of (Aib^{8,35})GLP-1(7-36)NH₂; 20 mM Ammonium acetate, pH=9.5; Kromasil C18 100-16; Acetonitrile/Methyl t-butyl ether (95:5 v:v). Purity and yield could be increased using Methyl t-butyl ether as organic modifier.

### DETAILED DESCRIPTION OF THE INVENTION

The present invention relates to a process for the purification of a GLP-1 peptide analogue applying reversed phase high performance liquid chromatography (RP-HPLC), said process comprising a first and a second chromatography step with a mixture of an aqueous buffer with an organic solvent for elution, characterized in that the organic solvent for the second chromatography step is acetonitrile and that the second chromatography step is performed using a basic buffer at a pH between 8.0 and 11.0.

An “aqueous buffer” is an aqueous solution containing a buffering agent that prevents a change in the pH. Depending on the buffering agent used the buffer can be acidic or basic.

The term “GLP-1 peptide analogue” encompasses the natural human glucagon-like peptide-1 (GLP-1) analogues GLP-1 (7-37) and GLP-1 (7-36)NH₂ and synthetic analogues of the GLP-1 peptide (GLP-1 analogues).

Particular GLP-1 analogues are the human GLP-1 analogue with the amino acid sequence according to SEQ ID No. 1:

His-Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Aib-Arg-NH₂, i.e. (Aib^{8,35}) GLP-1(7-36)NH₂, and further analogues as described in the PCT Publication WO 2000/34331. (Aib^{8,35}) GLP-1(7-36)NH₂ is of particular interest. The short form designates an analogue formally derived from natural human GLP-1 (1-37) by deleting the amino acid residues Nos. 1 to 6, amidating at the C-terminus and substituting the naturally occurring amino acid residues in position 8 (Ala) and 35 (Gly) by a-aminoisobutyric acid (Aib).

Suitable analogues of the GLP-1 peptide can further be selected from the group consisting of: GLP-1 (7-37), GLP-1 (7-36)NH₂, (Gly⁸) GLP-1(7-37), (Gly⁸) GLP-1(7-36), (Ser³⁴)GLP-1 (7-37), (Val⁸)GLP-1 (7-37), (Val⁸,Glu²²) GLP-1 (7-37), (N-ε-(γ-Glu(N-α-hexadecanoyl)))-Lys²⁶Arg³⁴-GLP-1(7-37) (Liraglutide) and D-Ala⁸Lys³⁷-(2-(2-(2-maleimidopropionamido(ethoxy)ethoxy)acetamide)) GLP-1 (7-37) (CJC-1131).

Still further analogues of the GLP-1 peptide can be the exendin analogues selected from exendin-3, exendin-4 (exenatide) having the amino acid sequence according to SEQ ID No. 2: His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH₂, exendin-4 acid, exendin-4 (1-30), exendin-4 (1-30) amide, exendin-4 (1-28), exendin-4 (1-28) amide, ¹⁴Leu,²⁵Phe exendin-4 amide and ¹⁴Leu,²⁵Phe exendin-4 (1-28) amide as well as AVE-0010, an exendin analogue having the amino acid sequence according to SEQ ID No. 3:

His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Ser-Lys-Lys-Lys-Lys-Lys-Lys-NH₂.

The second chromatography step is performed, as outlined above with acetonitrile as organic solvent and using a basic buffer at a pH between 8.0 and 11.0. IN a particular embodiment, the second chromatography step is performed at a pH of 9.0 to 10.0. In another particular embodiment, it is performed at a pH of 9.5+/−0.2.

In a particular embodiment of the present invention the acetonitrile is mixed with methyl t-butyl ether as organic modifier. For example, a mixture of acetonitrile and methyl t-butyl ether may be applied wherein said acetonitrile and said methyl t-butyl ether are present, respectively, in a ratio of from 99/1 (v/v) to 80/20 (v/v), particularly from 97.5/2.5 (v/v) to 90/10 (v/v), even more particularly 95/5 (v/v).

The basic buffer can be selected from commercial buffers known to the skilled in the art. In an embodiment of the present invention, the basic buffer is ammonium acetate or ammonium hydrogen carbonate.

The buffer concentration can be varied in a range between 10 to 25 mM, in particular 20 mM.

The first chromatography step is performed with acetonitrile as organic solvent and an acidic buffer at a pH between 1.0 and 4.0, more particularly at a pH between 2.0 and 3.0, at a pH of between 2.3 to 2.5, or at a pH of 2.5.

The acidic buffer can be selected from commercial buffers known to the skilled in the art. Ammonium phosphate was found to be particularly suitable. The buffer concentration can be varied in a range between 100 to 400 mM. In a particular embodiment, the buffer concentration is 300 mM.

The RP-HPLC is expediently performed using a silica gel sorbent as stationary phase.

Suitable silica gel types can be selected from, but are not limited to the following silica gel sorbents: Kromasil™ C18 100-16, Kromasil™ C18 100-10, Kromasil™ C8 100-16, Kromasil™ C4 100-16, Kromasil™ Phenyl 100-10, Kromasil™ C18 Eternity 100-5, Kromasil™ C4 Eternity 100-5, Chromatorex™ C18 SMB 100-15 HE, Chromatorex™ C8 SMB 100-15 HE, Chromatorex™ C4 SMB 100-15 HE, Daisopak™ SP 120-15 ODS-AP, Daisopak™ SP 120-10-C4-Bio, Daisopak™ SP 200-10-C4-Bio, Zeosphere™ C18 100-15, Zeosphere™ C8 100-15, Zeosphere™ C4 100-15, SepTech ST 150-10 C18, Luna C18 100-10, Gemini C18 110-10, YMC Triart C18 120-5 and YMC Triart C8 200-10.

The Kromasil™ silica gel types listed above were found to be particularly suitable. Alternatively the RP-HPLC can be performed by using polymeric based stationary phases. Suitable polymeric phases can be selected from, but are not limited to PLRP-S 100-10 or Amberchrom™ Profile XT20.

The RP-HPLC for both the first and the second chromatography step is run with mobile phase gradients, as a rule starting with a lower concentration of the organic solvent and over the elution time ending up with a higher concentration of the organic solvent. The elution parameters such as event time, mobile phase gradient and loading aspects can be varied by the skilled in the art in order to optimize the purification.

The fractions containing the purified (Aib^{8,35}) GLP-1(7-36)NH₂ can optionally be concentrated and subsequently lyophilized as described in PCT Publication WO 2007/147816. Alternatively the purified (Aib^{8,35}) GLP-1(7-36)NH₂ may be isolated from the RP-HPLC fractions by precipitation or by spray drying techniques known to the skilled in the art.

The following examples shall illustrate the process of the present invention in more detail without limiting the scope of it.

### EXAMPLES

### Example A

### Preparation of the Peptide

The crude peptide (Aib^{8,35})GLP-1(7-36)NH₂ can be prepared according to the methods described in WO 2007/147816 and WO 2009/074483 by producing three fragments and coupling these fragments in solution.

The purification involves a first pass chromatographic purification at a pH of 2.5, followed by a 2^{nd} pass at a pH of 9.5.

### Example B1

### RP-HPLC Technical Parameters

| | |
|---|---|
| HPLC System | Novasep Hipersep Lab LC 50 |
| Column | Novasep LC 60.500.VE100 (4.6 mm internal diameter) |
| Stationary Phase | RP silica gel (Kromasil 100-16-C18, 100 \acute{Å}, 16 μm) |
| | (Akzo Nobel) |
| Detection | UV (250 nm, 280 nm, 300 nm or 305 nm) |

### 1^{st} Chromatography Step:

Crude (Aib^{8,35})GLP-1(7-36)NH₂ was dissolved in water/acetonitrile/acetic acid (90/9/1 v/v/v) and loaded onto a HPLC column (loading up to 20 g/L, bed depth approx. 25 cm) and the purification program is initiated. Fractions are collected and may be diluted with water or diluted ammonium hydroxide solution.

TABLE 1
Parameters and Purification Program of 1^{st} Chromatography step:

| Parameter | Description |
|---|---|
| Eluent A | Aqueous ammonium phosphate (pH 2.5)/acetonitrile (80/20 v/v) |
| Eluent B | Aqueous acetic acid (0.1% w)/acetonitrile (25/75 v/v) |
| Eluent C | Aqueous ammonium phosphate (pH 2.5)/acetonitrile (60/40 v/v) |

| | | Composition | | | |
|---|---|---|---|---|---|
| Duration | Flow rate | Eluent A | Eluent B | Eluent C | |
| [min] | [mL/min] | [% (v/v)] | [% (v/v)] | [% (v/v)] | Remarks |
| 1.0 | 0.7 | 90.0 → 58.5 | 0 | 10.0 → 41.5 | Linear Gradient up to |
| | | | | | the start elution |
| | | | | | conditions. Duration |
| | | | | | may be adapted. |
| 40.0 | 0.7 | 58.5 → 46.5 | 0 | 41.5 → 53.5 | Linear gradient |
| 4.0 | 0.7 | 0 | 100 | 0 | Column flush |
| 7.0 | 0.7 | 90.0 | 0 | 10.0 | Conditioning |

Proportions of A and C may be varied in order to achieve a minimal retention for the main peak (peptide (Aib^{8,35})GLP-1(7-36)NH₂). The event time, gradient and loading aspects may be varied in order to optimize the purification. The pooled fractions are further purified by the conditions of 2^{nd} Chromatography.

### 2^{nd} Chromatography Step:

The pooled, diluted fractions from Chromatography 1 of (Aib^{8,35})GLP-1(7-36)NH₂ are loaded onto the HPLC column and the purification program (see examples for a 4.6 mm column in Table 2 is initiated.

TABLE 2
Parameters and Purification Program of 2^{nd} Chromatography step:

| Parameter | Description |
|---|---|
| Eluent D | Aqueous ammonium acetate 20 mM (pH 9.5 +/− 0.2) |
| Eluent E | Aqueous acetic acid (1% w)/acetonitrile (25/75 v/v) |
| Eluent F | Acetonitrile |

| | | Composition | | | |
|---|---|---|---|---|---|
| Duration | Flow rate | Eluent D | Eluent E | Eluent F | |
| [min] | [mL/min] | [% (v/v)] | [% (v/v)] | [% (v/v)] | Remarks |
| 1.0 | 0.7 | 90 → 76 | 0 | 10 → 24 | Gradient up to the start |
| | | | | | elution conditions. |
| | | | | | Duration may be |
| | | | | | adapted. |
| 40.0 | 0.7 | 76 → 56 | 0 | 24 → 44 | Linear gradient |
| 2.0 | 0.7 | 40 | 0 | 60 | Column flush |
| 2.0 | 0.7 | 0 | 100 | 0 | Flush and conditioning |
| | | | | | at acidic pH |
| 7.0 | 0.7 | 90 | 0 | 10.0 | Conditioning |

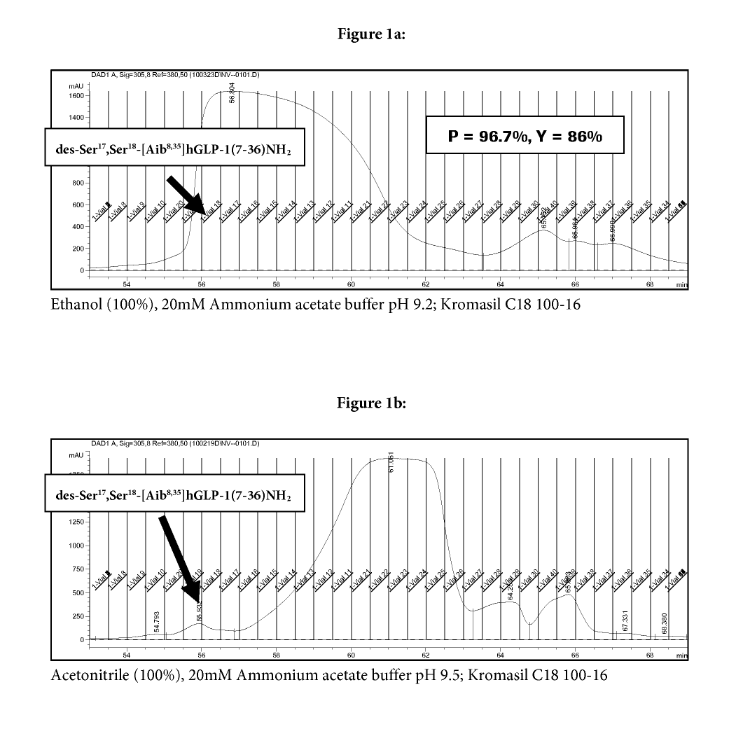

Calculated purity of (Aib^{8,35})GLP-1(7-36)NH₂ in the main fraction was 97.0%. The calculated yield was 87% (see FIG. 1b, 2a).

### Example B2

The procedure of Example B1 was repeated with the exception that for the second chromatography step an ammonium hydrogen carbonate buffer (20 mM (pH 9.5+/−0.2) was used. Calculated purity of (Aib^{8,35})GLP-1(7-36)NH₂ in the main fraction was 97.2%. The calculated yield was 93%.

### Example B3

The procedure of Example B1 was repeated with the exception that for the second chromatography step acetonitrile was replaced by a mixture of acetonitrile/methyl t-butyl ether 95:5.

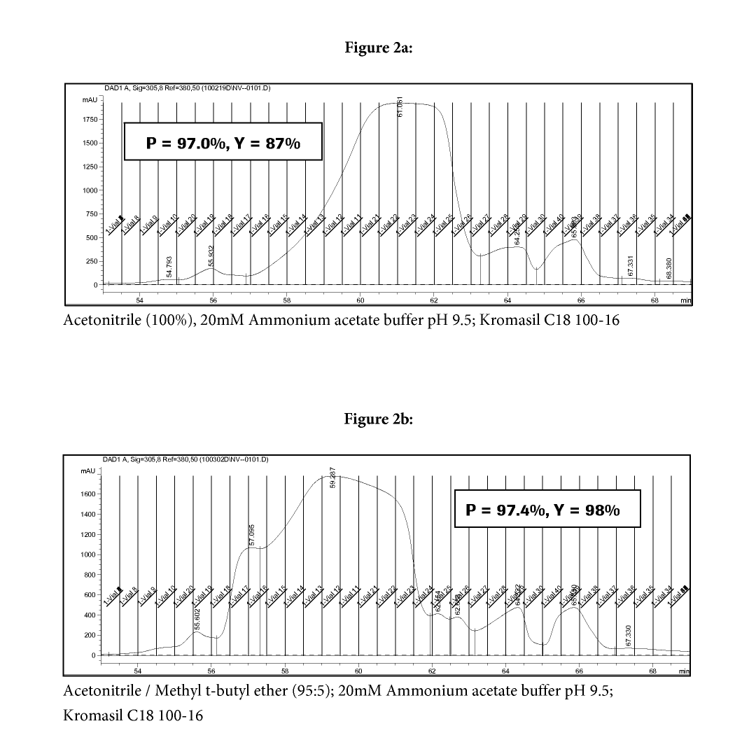

Calculated purity of (Aib^{8,35})GLP-1(7-36)NH₂ in the main fraction was 97.4%. The calculated yield was 98% (see FIG. 2b).

### Example B4

The procedure of Example B1 was repeated applying the following parameters.

| Parameter | Description | | | | |
|---|---|---|---|---|---|
| Eluent G | Aqueous ammonium acetate 20 mM (pH 9.5 +/− 0.2)/acetonitrile (80:20 v/v) | | | | |
| Eluent H | Aqueous acetic acid (0.1% w)/acetonitrile (25/75 v/v) | | | | |
| Eluent I | Aqueous ammonium acetate 20 mM (pH 9.5 +/− 0.2)/acetonitrile (60:40 v/v) | | | | |
| | | Composition | | | |
| Duration | Flow rate | Eluent G | Eluent H | Eluent I | |
| [min] | [mL/min] | [% (v/v)] | [% (v/v)] | [% (v/v)] | Remarks |
| 1.0 | 0.7 | 90.0 → 57.0 | 0 | 10.0 → 43.0 | Linear Gradient |
| | | | | | up to the start |
| | | | | | elution |
| | | | | | conditions. |
| | | | | | Duration may be |
| | | | | | adapted. |
| 40.0 | 0.7 | 57.0 → 27.0 | 0 | 43.0 → 73.0 | Linear gradient |
| 2.0 | 0.7 | 0 | 0 | 100 | Column flush |
| 2.0 | 0.7 | 0 | 100 | 0 | Flush and |
| | | | | | conditioning at |
| | | | | | acidic pH |
| 7.0 | 0.7 | 90 | 0 | 10.0 | Conditioning |

Calculated purity of (Aib^{8,35})GLP-1(7-36)NH₂ in the main fraction was 97.1%. The calculated yield was 99%.

### Example B5 (Comparison)

The procedure of Example B1 was repeated with the exception that for the second chromatography step acetonitrile was replaced by ethanol.

Calculated purity of (Aib^{8,35})GLP-1(7-36)NH₂ in the main fraction was 96.7%. The calculated yield was 86%. The main fraction contained des-Ser¹⁷, Ser¹⁸-[Aib^{8,35}]hGLP-1(7-36)NH₂ as impurity (see FIG. 1a).

1. A process for the purification of a GLP-1 peptide analogue applying reversed phase high performance liquid chromatography (RP-HPLC) comprising a first and a second chromatography step with a mixture of an aqueous buffer with an organic solvent for elution, characterized in that the organic solvent for the second chromatography step is acetonitrile and that the second chromatography step is performed using a basic buffer at a pH between 8.0 and 11.0.

2. A process according to claim 1, wherein said acetonitrile is mixed with methyl t-butyl ether as an organic modifier.

3. A process according to claim 2, wherein a mixture of acetonitrile and methyl t-butyl ether is applied and said acetonitrile and said methyl t-butyl ether are present, respectively, in ratio of from 99/1 (v/v) to 80/20 (v/v).

4. A process according to claim 1, wherein said basic buffer is ammonium acetate or ammonium hydrogencarbonate.

5. A process according to any one of claim 1, wherein said basic buffer is applied in a concentration of 10 mMol to 25 mMol.

6. A process according to claim 1, wherein the aqueous organic solvent for the first chromatography step is acetonitrile and the first chromatography is performed using an acidic buffer at a pH between 1.0 and 4.0.

7. A process according to claim 6, wherein said acidic buffer is ammonium phosphate.

8. A process according to claim 1, wherein said RP-HPLC is performed using a silica gel sorbent as stationary phase.

9. A process according to claim 1, wherein the GLP-1 peptide analogue is selected from the group consisting of GLP-1 (7-37), GLP-1 (7-36)NH₂, (Gly⁸) GLP-1(7-37), (Gly⁸) GLP-1(7-36), (Ser³⁴)GLP-1 (7-37), (Val⁸)GLP-1 (7-37), (Val⁸,Glu²²) GLP-1 (7-37), (Aib^{8,35})hGLP-1(₇₋₃₆)NH₂, (N-ε-(γ-Glu(N-α-hexadecanoyl)))-Lys²⁶Arg³⁴-GLP-1(7-37), D-Ala⁸Lys³⁷-(2-(2-(2-maleimidopropionamido(ethoxy)ethoxy)acetamide)) GLP-1 (7-37), exendin-3, exendin-4, exendin-4 acid, exendin-4 (1-30), exendin-4 (1-30) amide, exendin-4 (1-28), exendin-4 (1-28) amide, ¹⁴Leu,²⁵Phe exendin-4 amide and ¹⁴Leu,²⁵Phe exendin-4 (1-28) amide and AVE-0010.

10. A process according to claim 1, wherein said GLP-1 peptide analogue is (Aib^{8,35})hGLP-1(7-36)NH₂.

11. A purified GLP-1 peptide analogue as purified using a process according to claim 1.
